(19) United States
(12) Patent Application Publication
(10) Pub. No.: US 2006/0129227 A1
(43) Pub. Date: Jun. 15, 2006

(54) Auxetic tubular liners

(75) Inventor: Rudy Hengelmolen, Felbridge (GB)

Correspondence Address: ROTHWELL, FIGG, ERNST & MANBECK, P.C., 1425 K STREET, N.W., SUITE 800, WASHINGTON, DC, 20005, US

(73) Assignee: Auxetica Limited, London (GB)

(21) Appl. No.: 10/522,889

(22) PCT Filed: Aug. 4, 2003
(86) PCT No.: PCT/GB03/03393

§ 371 Date: Aug. 18, 2005

Related U.S. Application Data
(60) Provisional application No. 60/409,008, filed on Sep. 9, 2002.

(30) Foreign Application Priority Data
Aug. 2, 2002 (GB) 0217973.7

(51) Int. Cl. A61F 2/90; B44C 1/22; A61F 2/84

(52) U.S. Cl. 623/1.16; 623/1.11; 216/8

(57) ABSTRACT

The present invention concerns tubular liners for insertion into a duct, said tubular liners defining first and second ends and a lumen, both of said first and second ends being open, such that fluid flow can occur through said tubular liners from said first end to said second end, characterised in that said liners comprises an auxetic material. Also provided are auxetic tubular liners having a range of specific structures, together with methods of lining ducts using said tubular liners, and apparatus for same.

This invention in its broader aspects relates to components for use in lining ducts. One specific application of the invention is concerned with stents for use in counteracting obstructions or narrowing in in vivo ducts such as blood vessels, bile ducts in the liver or pancreas, gastrointestinal tubes such as the esophagus, urethra and ureter ducts and pulmonary passageways.

According to the present invention there is provided a tubular liner for insertion into a duct, said tubular liner defining first and second ends and a lumen, both of said first and second ends being open, such that fluid flow can occur through said tubular liner from said first end to said second end, characterised in that said liner comprises an auxetic material.

References to auxetic material herein include materials which are intrinsically auxetic and materials which have been rendered auxetic (as discussed hereinafter).

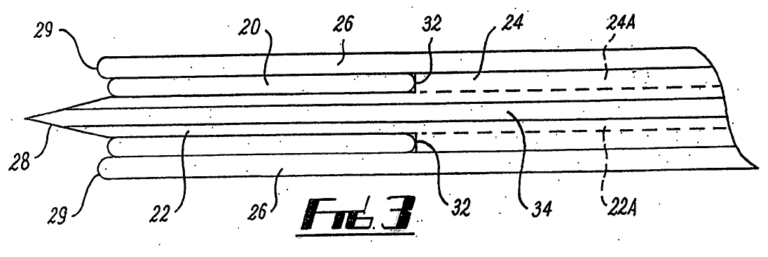

Conventional materials have a positive Poisson ratio, i.e. when stretched in one direction they tend to become thinner in a direction lateral to the direction of elongation—Poisson's ratio is the ratio of the lateral contraction per unit breadth, to the longitudinal extension per unit length when a piece of material is stretched. Auxetic materials exhibit a negative Poisson ratio in that they expand in a direction perpendicular to the direction of stretching. Auxetic materials also have the capacity for formation into doubly curved or dome shaped surfaces due to the synclastic property of auxetic materials, a property which is described in for instance WO 99/22838 with reference to FIG. 2(b) thereof.

Thus with the tubular duct liners of the present invention, when they are radially compressed, they become shorter, whereas when they are radially expanded, they increase in length.

The auxetic material may be a synthetic auxetic material and may have a macroscopic or microscopic auxetic structure.

The auxetic material may be polymeric.

The liner may be in the form of a metallic, auxetic mesh structure.

The auxetic material may be of a porous nature.

The auxetic material forming the liner may comprise a biodegradable polymer or polymers. In the case of a stent comprising or consisting of a tubular liner of the present invention, this may be advantageous in allowing breakdown of the stent in the body over the course of time.

Examples of biodegradable polymers include polyglycollic acid and its copolymers, polylactic acid (both D and L isomers) and their copolymers, poly-β-hydroxybutyrate, poly-β-hydroxypropionate, poly-ε-caprolactone, poly-δ-valerolactone, poly(methylmethacrylate-co-N-vinylpyrrolidone), polyvinyl alcohol, polyanhydrides, poly-ortho-esters, and polyphosphazenes. Of particular use are polyglycollic acid (PGA), as well as its copolymers and the isomeric polylactic acids, PLLA and PDLLA, together with their copolymers. Polymers and copolymers of ε-caprolactone are also highly useful. Other biodegradable materials are detailed in: The Chemistry of Medical and Dental Materials, JW Nicholson, Royal Society of Chemistry, ISBN: 0854045724.

An auxetic material for use in the invention may be selected from any suitable material, including the known auxetic materials mentioned below.

Synthetic auxetic materials are known from for example U.S. Pat. No. 4,668,557 which discloses preparation as an open-celled polymeric foam, negative Poisson ratio properties being secured by mechanical deformation of the foam by compression. Auxetic materials may also be in the form of microporous polymers, polymer gels, and macroscopic cellular structures (e.g. structures comprising re-entrant “bow tie” or inverted hexagon units). A polymeric material is disclosed in WO 91/01210, the material having an auxetic microstructure of fibrils connected at nodes and being obtained by compacting polymer particles at elevated temperatures and pressures, sintering and then deforming the compacted polymer by extrusion through a die to produce a cylindrical rod of auxetic material. WO 00/53830 discloses an auxetic polymeric material which is of filamentary or fibrous form which is produced by cohering and extruding thermoformable particulate material, cohesion and extrusion being effected with spinning so that an auxetic microstructure of fibrils and nodes can be obtained without requiring separate sintering and compaction stages. Auxetic materials have for example been produced of polytetrafluoroethylene, polyethylene, nylon and polypropylene. Particularly useful materials for the auxetic tubular liners of the present invention are nylon, polyurethanes and polyesters.

Although the possibility of the stent being metallic is not excluded, production of the stent using a polymer of suitable tissue-compatibility is preferred since it eliminates the risk, which can occur where metallic stents are deployed, of chemical reaction between the metal and its immediate environment (i.e. dilated plaque tissue).

A liner in accordance with the invention typically comprises an auxetic material produced by:
(a) machining appropriate geometry, e.g. inverted microhexagons, into the structure; or
(b) processing, i.e. compression and subsequent deformation of polymeric powder particles into a tubular form under controlled conditions of pressure and temperature; or
(c) a combination of processing and subsequent micromachining.

Appropriate geometry such as inverted microhexagons can be machined into the material which forms or is to form the tubular liner using an excimer laser system. Machining by means of excimer laser technology allows feature sizes from about 4 mm to about 2 micron to be etched into a wide variety of materials and features of the order of 10 micron in size or larger can be drilled through the entire thickness of a substrate. The structures detailed below in the specific embodiments of the invention have been manufactured using an excimer laser.

The tubular liner may comprise a stent for insertion, e.g. with the aid of a catheter, into an in vivo duct, examples of which are given hereinbefore.

The tubular liner may be sufficiently flexible that, by virtue of the synclastic property of auxetic materials, it can be readily turned inside out within the confines of a duct, e.g. a blood vessel or other in vivo duct, in which it is to be installed or implanted.

Prior art coronary stents are made of, or based on, metal and are either self-expandable or capable of undergoing plastic deformation (i.e. they only deform when pressurised and cannot regain their original shape in the absence of an external force or pressure).

Both types of prior art stent are deployed either during or following balloon angioplasty, which involves dilation of plaque deposit on the inner side of an artery wall by briefly inflating a balloon to a relatively high pressure (most plaques break at between 4-6 atmospheres).

The application of balloon angioplasty inherently causes tissue damage of the stented region as well as near the ends of the stent. The risk of over-dilation with this technique is high and metal stents that expand through plastic deformation will, in the case of over-dilation, keep the artery over-stretched, which is linked to restenosis—the in-growth of scar tissue that is likely to block the artery again gradually within months. Restonosis specifically occurs close to the end sections of a stent, where stress concentrations are greatest relative to other stented parts of the artery.

Other more compliant self-expandable metal stents are available which, compared to balloon-expandable stents, reduce to some extent the degree of permanent over-stretching of the artery wall. However, their mesh structure generally requires smaller unit cells in order to keep the artery open, but this typically inhibits longitudinal flexibility of the stent. The auxetic structures of the tubular liners of the present invention provide for enhanced longitudinal and localised radial flexibility.

The present invention seeks to overcome these prior art disadvantages, and particularly to provide tubular liners which provide increased counteraction upon compression, but whose longitudinal or localised radial flexibility is not inhibited or reduced under typical in vivo conditions as a result.

As mentioned above, one useful form of tubular liner of the present invention uses a geometry of inverted hexagons in order to effect auxetic properties in a tubular structure which would otherwise not be auxetic. These “inverted hexagons” are not “regular” hexagons and instead essentially comprise a hexagon having first and second sides opposite and parallel to one another, and then third, fourth, fifth and sixth inwardly-inclined sides joining them. The present inventor has found that by linking chains of such inverted hexagons together via their third, fourth, fifth and/or sixth sides, then an auxetic structure can be created. Obviously, it is possible to incorporate into such structures inverted hexagons which are linked together via the vertices of their first and second sides, although this may result in non-auxetic regions whilst still retaining the overall auxetic properties.

Thus a tubular liner according to the present invention, said tubular liner defining a longitudinal axis between said first and second ends, can have a structure comprising a plurality of adjacent radial loops arranged about said tubular liner, each radial loop comprising a plurality of interconnected hexagons having:

(i) first and second sides parallel with and opposite to one another;

(ii) third and fourth sides dependent from said first side; and

(iii) fifth and sixth sides dependent from said second side;

said third side being connected to said fifth side at a first vertex, and said fourth side being connected to said sixth side at a second vertex;

said first side of each hexagon making an internal angle of less than 90 degrees with each of said third and fourth sides, and said second side making an internal angle of less than 90 degrees with each of said fifth and sixth sides;

said first and second sides of said hexagons being oriented in said longitudinal axis;

each hexagon being connected to first and second adjacent hexagons, said first side of each hexagon comprising a second side of said first adjacent hexagon, and said second side comprising a first side of said second adjacent hexagon;

each radial loop being connected to at least a first adjacent radial loop, each pair of first and second adjacent radial loops being connected by a plurality of connecting members.

The plurality of connecting members may be between said third and fifth sides of said plurality of hexagons of said first adjacent radial loop and said fourth and sixth sides of said plurality of hexagons of said second adjacent radial loop.

The connecting members may be other than between the vertices of said first and second sides.

In certain embodiments of the present invention, it may be desirable to arrange the adjacent loops of hexagons such that they are offset relative to one another. For example, it may be desirable to arrange a first loop so that the vertices of its first and second sides with its third and fifth sides are proximal to the vertices made between the fourth and sixth sides of hexagons of a second loop. For example, a connecting member may join the first and second loops by connecting the vertices of the first and second sides of the first loop (made with its third and fifth sides) to the vertex made between the fourth and sixth sides of the hexagons of the second loop.

Alternatively, the connecting members can for example be between said first vertex of said hexagons of said first loop and said second vertex of said hexagons of said second loop.

Examples of such tubular liners are detailed below. Properties of the tubular liner, including the extent of its auxetic nature, can be modified depending upon the exact construction of the inverted hexagons. The above general structure is particularly useful where it is desired to have a tubular liner which is able to be expanded and compressed radially.

In particular, said connecting members may be between said first vertex of said hexagons of said first loop and said second vertex of said hexagons of said second loop.

The expandable and contractible nature of the material means that it can also be particularly useful when seeking to line a vessel which is partially blocked, for example a blood vessel. Since compression of the tubular liners is ultimately limited by the ability of the inverted hexagons to compress, there is as a result a maximum extent to which compression can be effected (i.e. the tubular liners have a minimum radius), and this is dictated by the construction of the inverted hexagons. As compression takes place, the tubular liner becomes more rigid in its structure at the point or region of compression and more resistant to deformation, the degree of which is controlled by the structure of the tubular liner (e.g. first and second sides perpendicular to the longitudinal axis, or parallel to it). For example, increasing the length of the connecting members increases the flexibility of the tubular liner.

If used as a stent to line a blood vessel which has a partial blockage (for example an artery which has a plaque formation) the tubular liners of the present invention are able to accommodate such plaques by reducing in diameter locally to the plaque (or other surface feature of the duct). As the tubular liner is compressed, the stronger and more resistant to compression it becomes. This contrasts to conventional prior art vascular stents which typically have very limited local compressibility yet risk eventual collapse (recoil in use). Prior art stents designed to overcome recoil will inherently risk significant over-expansion of the vessel walls when using an ancillary device to expand to accommodate the stent (and build up contact stress in the vessel).

The tubular liners of the present invention can be structured to ensure that fluid flow can be achieved along their length by having a minimum radius to which they can be compressed. Prior art stents also fail to show the relative increase in strength upon compression achieved by the auxetic tubular liners of the present invention. In addition, the structures of the present invention can be made highly flexible, even when compressed. In addition, the use of conventional means for removing or dilating obstructions in vessels (such as ancillary devices for example in balloon angioplasty) results in inherent injury of diseased tissue, whereas contact stress is also observed, resulting in a gradual re-blocking at the site at which a stent is inserted. (Moore J Jr, Berry J L, Ann Biomed Eng. 2002 April;30(4):498-508; PMID: 12086001). The tubular liners of the present invention allow the minimisation of contact stress between a vascular (e.g. coronary) stent and diseased tissue by providing a support structure (the stent) which allows the vessel to conform to its natural flexibility, and also provides a less invasive route to dilation of blockages than prior art means.

As well as the above tubular liner structures using inverted hexagons (in which the first and second parallel sides are oriented in the longitudinal axis of the tubular liner), structures can also be made in which the first and second parallel sides are oriented perpendicular to the longitudinal axis of the tubular liner. These structures whilst also being auxetic can be manufactured such that they are capable of little radial compression or expansion, yet are capable of substantial longitudinal compression or expansion.

Thus a tubular liner according to the present invention, said tubular liner defining a longitudinal axis between said first and second ends, can have a structure comprising a plurality of longitudinally elongate strips of interconnected hexagons oriented along said longitudinal axis of said tubular liner, each longitudinally elongate strip comprising a plurality of interconnected hexagons having:

(i) first and second sides parallel with and opposite to one another;

(ii) third and fourth sides dependent from said first side; and

(iii) fifth and sixth sides dependent from said second side;

said third side being connected to said fifth side at a first vertex, and said fourth side being connected to said sixth side at a second vertex;

said first side of each hexagon making an internal angle of less than 90 degrees with each of said third and fourth sides, and said second side making an internal angle of less than 90 degrees with each of said fifth and sixth sides;

said first and second sides of said hexagons being oriented perpendicular to said longitudinal axis;

each hexagon being connected to at least a first adjacent hexagon, said first side of each hexagon comprising a second side of said first adjacent hexagon, and said second side comprising a first side of any second adjacent hexagon;

each longitudinally elongate strip being connected to first and second radially adjacent longitudinally elongate strips by a plurality of connecting members.

The plurality of connecting members may be between:
(a) said third and fifth sides of said plurality of hexagons of said longitudinally elongate strip and said fourth and sixth sides of said plurality of hexagons of said first radially adjacent longitudinally elongate strip; and
(b) said fourth and sixth sides of said plurality of hexagons of said longitudinally elongate strip and said third and fifth sides of said plurality of hexagons of said second radially adjacent longitudinally elongate strip.

The connecting members may be other than between the vertices of said first and second sides.

As for the looped arrangements of hexagons, the strips of hexagons may be offset relative to one-another and adjacent strips may be joined by connecting members appropriately.

In such a tubular liner, the connecting members may be between:

(a) said first vertex of said hexagons of a given longitudinally elongate strip and said second vertex of said hexagons of a first radially adjacent longitudinally elongate strip of hexagons; and

(b) said second vertex of said hexagons of said given longitudinally elongate strip and said first vertex of said hexagons of a second radially adjacent longitudinally elongate strip of hexagons.

In the various embodiments of the present invention which use polygons such as hexagons connected together forming either adjacent longitudinally elongate strips or adjacent radial loops, the connecting member can be shaped as desired, so long as the eventual structure defined is auxetic. For example, the connecting members can be straight, curved or angled.

The simplest possible shape is a straight one, and a straight connecting member can be arranged parallel to the first and second sides of a hexagon to which it is connected. As mentioned above, straight connectors can be between first and second vertices of adjacent hexagons, or they can be between e.g. third and fifth or fourth and sixth sides of adjacent hexagons. Alternatively, a straight connecting member can be arranged at an angle to the first and second sides of a hexagon to which it is connected.

Alternative structures include curved and angled structures. As mentioned above, the requirement is that the final structure incorporating the connecting members is auxetic. Therefore, in the above embodiments all of the hexagons cannot be connected by connecting members between vertices of first or second sides of adjacent hexagons.

As well as the above “inverted hexagon” structures, the use of other auxetic structures falls within the scope of the present invention. In particular, the first and second sides mentioned above which are parallel to and opposite one another can be replaced with e.g. sides having relatively inflexible branched sections. Thus for example first and second sides can be replaced with a first side having first and second vertices, and with first and second arms extending from each of the first and second vertices, each of the first and second arms making an internal angle with the first side of between 90 and 180 degrees. For example, internal angles of between 91 and 179 degrees can be made, e.g. 125, 130, 135, 140, 145 or 150 degrees. Third, fourth, fifth and sixth sides can then depend from the first and second arms of the first and second sides, thus completing the polygons. By making the third, fourth, fifth and sixth sides relatively flexible compared to the first and second sides and the first and second arms, the auxetic properties of the structures and tubular liners of the present invention are ensured. Examples of such structures are given below.

According to the present invention there is also provided an assembly for use in lining a section of duct, said assembly comprising:

(i) a tubular liner according to the present invention;

(ii) a mandrel upon which said auxetic tubular liner is located; and

(iii) a sleeve surrounding said mandrel and auxetic tubular liner, said sleeve having an open end;

said mandrel being movable relative to said sleeve.

Also provided according to the present invention is the use of a tubular liner according to the present invention in the manufacture of an assembly according to the present invention for use in lining a section of duct.

Also provided according to the present invention is a method of inserting a tubular liner according to the present invention into a duct, said tubular liner defining first and second faces, said first face facing said lumen, said second face facing away from said lumen, said method comprising the steps of:

(i) locating said tubular liner on a mandrel surrounded by a sleeve to define an assembly, said sleeve having an open end;

(ii) passing said assembly into said duct;

(iii) moving said mandrel relative to said sleeve so as to cause said tubular liner to be displaced through said sleeve open end such that said tubular liner folds back over said sleeve and inverts within the confines of said duct such that said second face faces said lumen of said inverted tubular liner and said first face faces away from said lumen of said inverted tubular liner;

(iv) withdrawing said sleeve and said mandrel from said duct, leaving said inverted tubular liner in situ.

The open end of the sleeve through which the liner is displaced may have a convexly curved end face to facilitate folding back of the liner over the sleeve and pressure transduction in the lateral direction.

The mandrel may be provided with an ancillary element for use, for example, in softening up and/or pre-dilation of material deposited within the duct. Alternatively or additionally, the mandrel may be provided with a laser radiation transmission path, e.g. a fibre optic, to allow laser radiation to be directed into the duct, for instance to treat clogged or plaque-filled ducts.

The mandrel may define a leading end portion and be provided with a passageway or passageways in communication with said leading end portion of the mandrel to allow fluids to be withdrawn from the duct.

The arrangement may be such that, during insertion of the liner, fluid flow (e.g. blood flow) through the assembly is possible. This may be achieved for instance by providing one or more apertures or slits in the sleeve as well as in the mandrel so that fluid flow can take place from one side of the assembly to the other via a pathway extending from said one side, around the outside of the assembly, through the apertures or slits in the sleeve and mandrel and to the other side of the assembly. For instance, in the case of a stent, during insertion of the stent such an arrangement may allow blood flow from a point upstream of a narrowing or obstruction to a point downstream thereof.

The mandrel may include a portion which may be radially expanded. This may serve to facilitate dilation of obstructions such as plaque in the duct, e.g. during deployment of the liner, and/or facilitate “back folding” of the leading part of the liner around the sleeve, and lateral pressure transduction.

The liner may be adapted for use in the delivery of drugs or other beneficial agents, e.g to the site of narrowing or obstruction in an in vivo duct such as an artery. Fabrication of the liner from biodegradable materials may be advantageous in this context because of the possibility of exploiting biodegradability, both in terms of allowing degradation of the tubular liner over time, and in terms of controlling the release profile of such agents from the liner.

Suitable applications for the auxetic tubular liners of the present invention include their use as stents, including as elongate stents (for example having a length of at least 3 cm) which can be used to strengthen an elongate section of duct such as a blood vessel. The tubular liners are also useful in surgical procedures, for example when a surgeon desires to operate upon a diseased (e.g. ballooned) section of a blood vessel (for example an artery) whilst not having to block blood flow through the vessel. By inserting a tubular liner of the present invention in the vessel co-extensive with the diseased region, and extending beyond both ends of the diseased section, a substantial route for blood flow between the ends of the diseased section can be provided. During surgery upon the diseased section then in the event of the diseased section of the vessel rupturing, the tubular liner allows the maintenance of blood flow between the ends of the diseased section, reducing the need for e.g. acute clamping to block blood flow and allowing surgery to proceed to effect whatever repairs are necessary to the blood vessel.

The tubular liners of the present invention can also be provided with polygonal shapes (such as “inverted hexagons”) of varying size. For example, in the case of a stent for use in a blood vessel which is connected to branching vessels, the tubular liner could be provided with large polygons (e.g. hexagons) at a point at which it is desired to allow the free-flow of blood into and/or out of the tubular liner, and with smaller hexagons at other points.

As discussed above, in the case of hexagons (and also other polygons), different orientations of the polygons result in different properties for the tubular liner—in the case of hexagons, those having the first and second sides oriented in the longitudinal axis of the tubular liner are typically highly radially compressible compared to their longitudinal compressibility, and those with their first and second sides oriented perpendicular to the longitudinal axis of the tubular liner are highly longitudinally compressible compared to their radial compressibility.

The stent may, if desired, be used as a vehicle for delivery of drugs or other beneficial agents to the site of a diseased vessel (e.g. narrowed or obstructed), e.g. wound-healing agents or DNA materials such as oligopeptides. Such agents may be incorporated in the porous auxetic material, e.g. by chemical and/or physical fixation. The drug or other agent can be incorporated into the interstitial voids or it can be introduced by blending into polymeric particles which are to be used in production of the stent, either by processing into a microporous auxetic tube or into a non-auxetic tube which is subsequently transformed into an auxetic scaffolding, e.g. by micromachining, or the drug can be absorbed by, or adsorbed onto, a finished structure. Other uses of drugs are the coating of the outer (facing away from the lumen of the tubular liner) and inner (facing towards the lumen of the tubular liner) surfaces of the tubular liner. For example, the outer surface can be coated with a cell pacifier, whereas the inner surface can be coated with an anticoagulant such as heparin.

The invention will be further apparent from the following description, with reference to the several figures of the accompanying drawings, which show, by way of example only, forms of auxetic tubular liners.

Of the Figures:

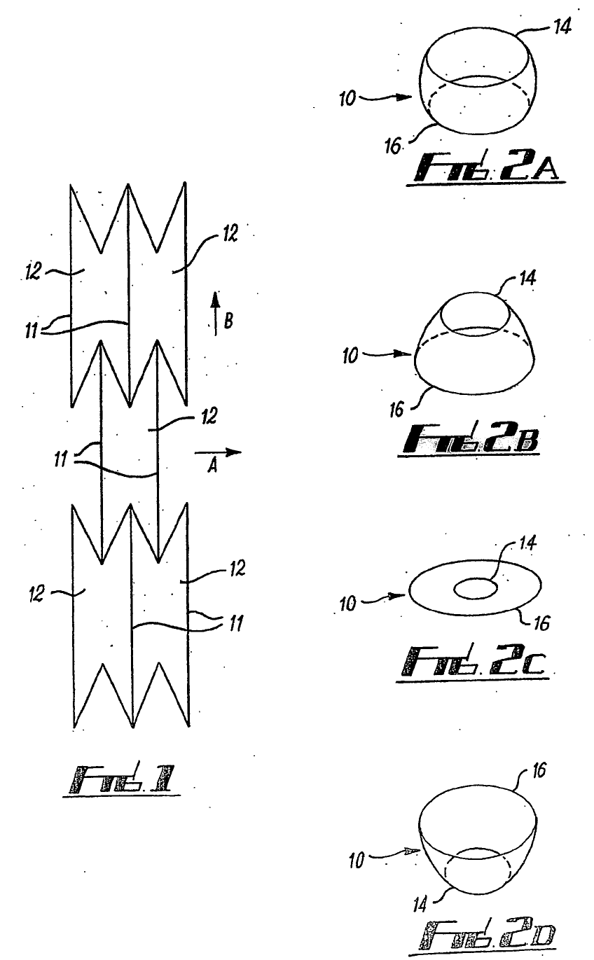

FIG. 1 shows the geometrical features of an auxetic material which may be made use of in a tubular liner or stent in accordance with the present invention;

FIG. 2 shows (FIGS. 2A to 2D) the inversion of an auxetic tubular structure of relatively short length;

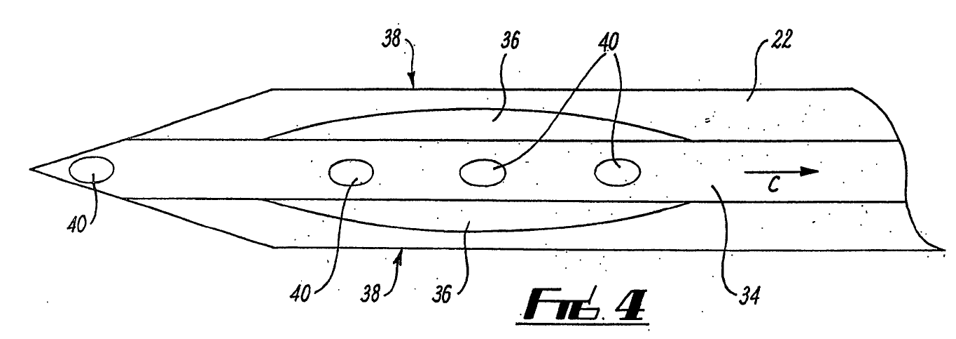

FIG. 3 shows a sectional view of an assembly for use in implanting a stent within an in vivo duct such as a blood vessel;

FIG. 4 shows an enlarged view showing details of the mandrel of the assembly shown in FIG. 3;

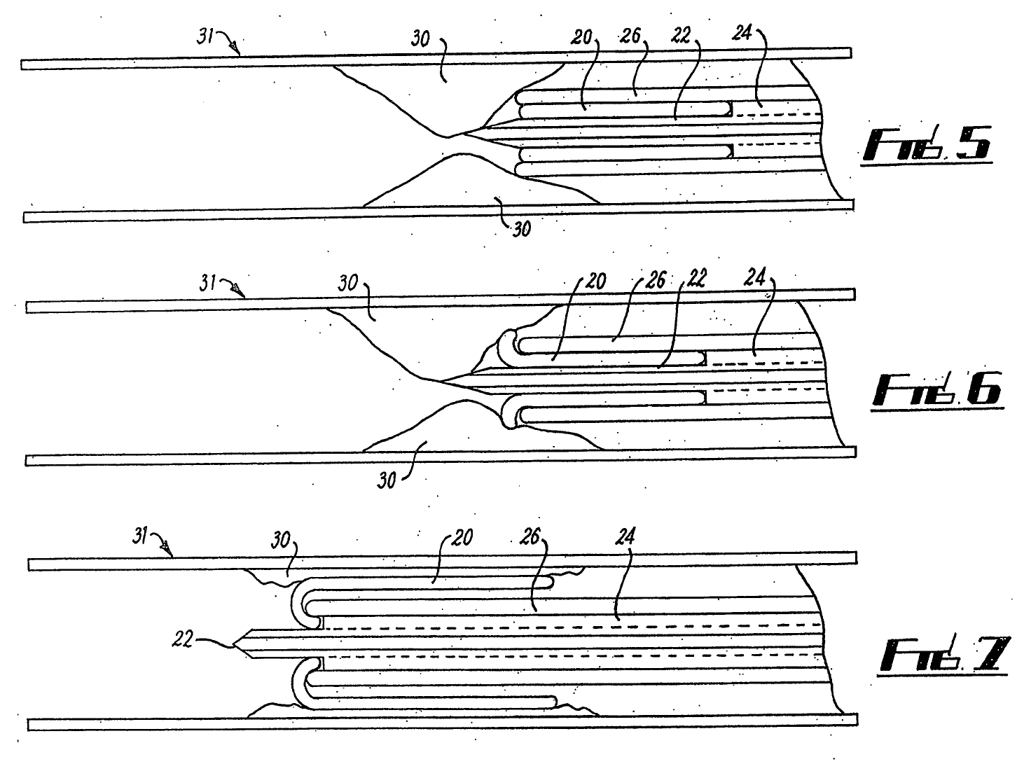

FIGS. 5-7 are views showing successive stages in the use of the assembly to implant the stent within a blood vessel or the like;

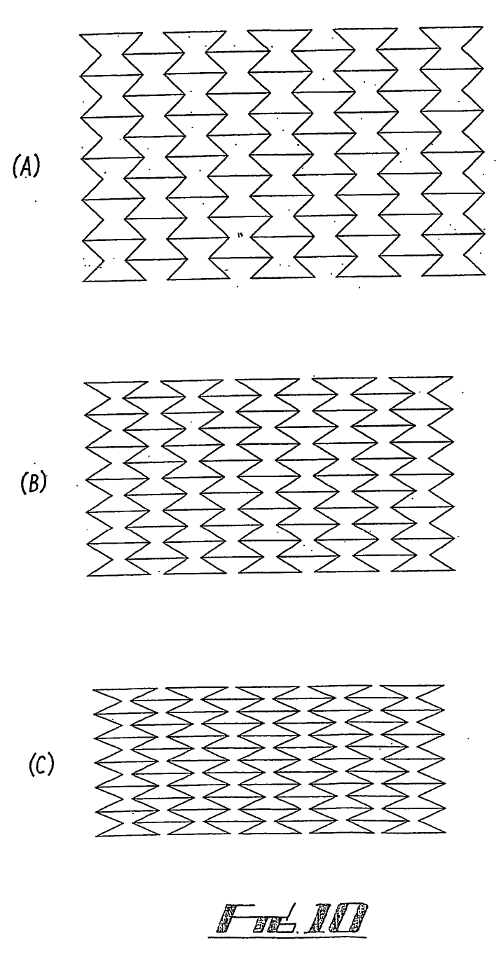

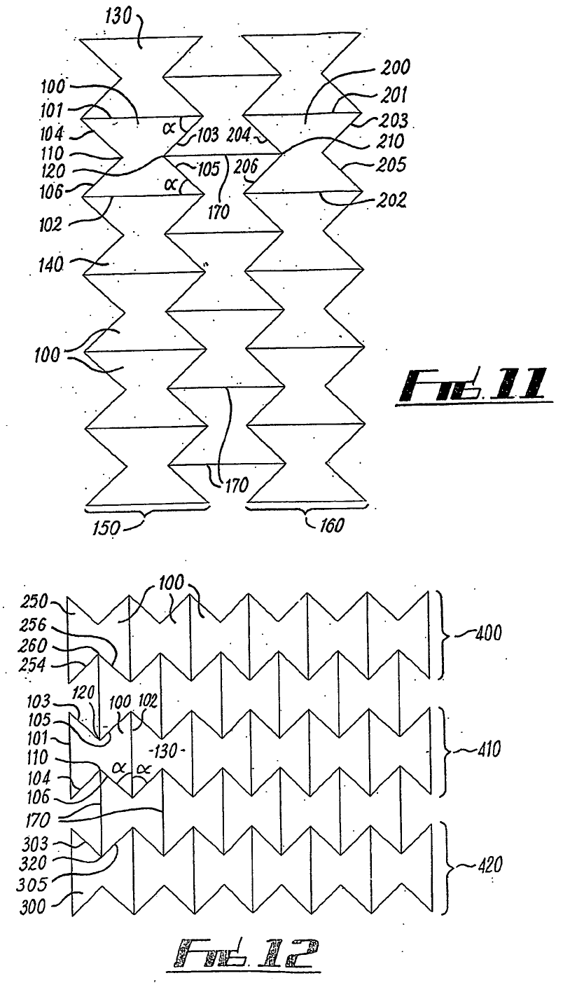

FIGS. 8-9 are views illustrating transfer of the stent on to the mandrel during the course of preparing the assembly of FIG. 3;

FIG. 10 shows the effect compression of a section of auxetic tubular material;

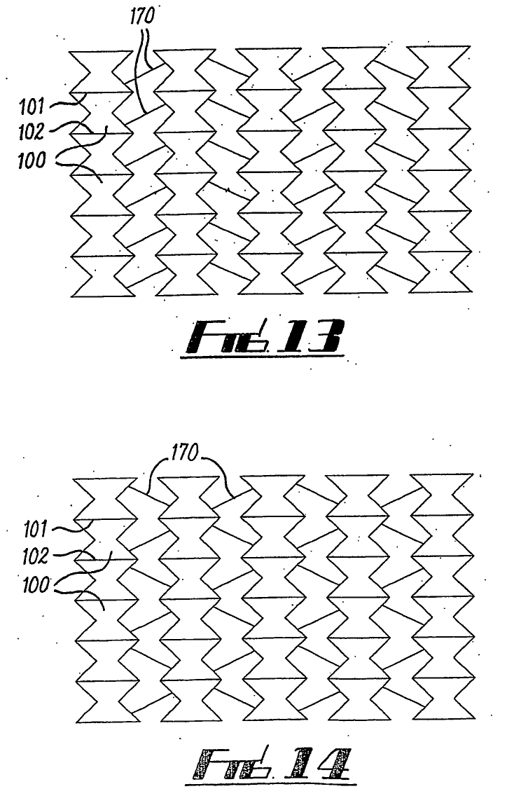

FIG. 11 shows a section of a first auxetic tubular liner having an “inverted hexagon” structure.

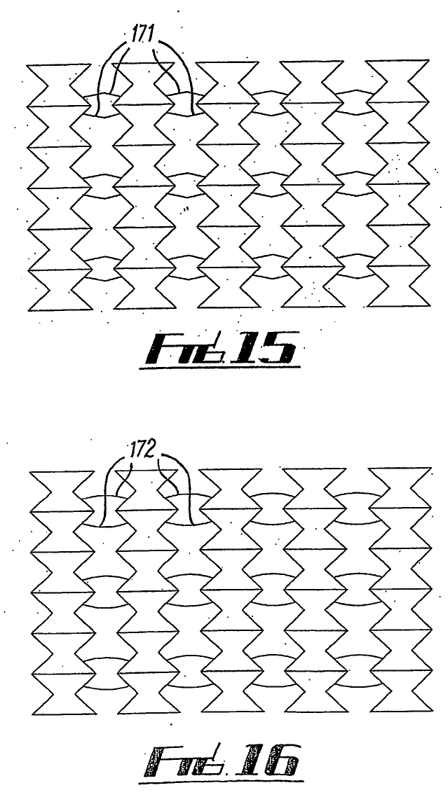

FIG. 12 shows a section of a second auxetic tubular liner having an “inverted hexagon” structure perpendicularly arranged relative to the structure of FIG. 11;

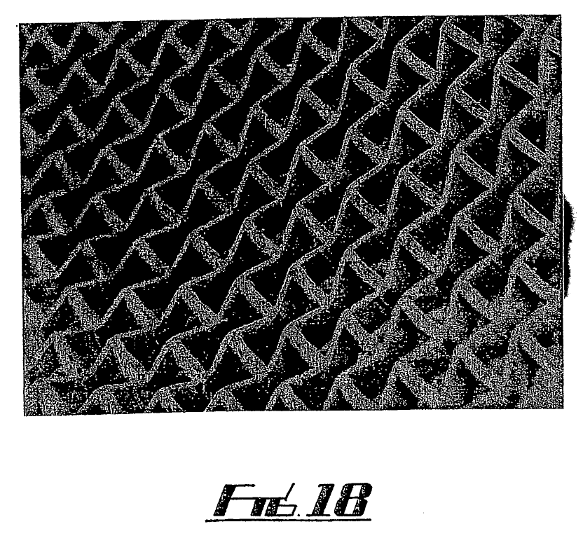

FIGS. 13-16 show alternative embodiments with an auxetic structure comprised of hexagons and (FIGS. 13,14) straight connecting members at an angle to the parallel first and second sides, angled connecting members (FIG. 15) and curved connecting members (FIG. 16);

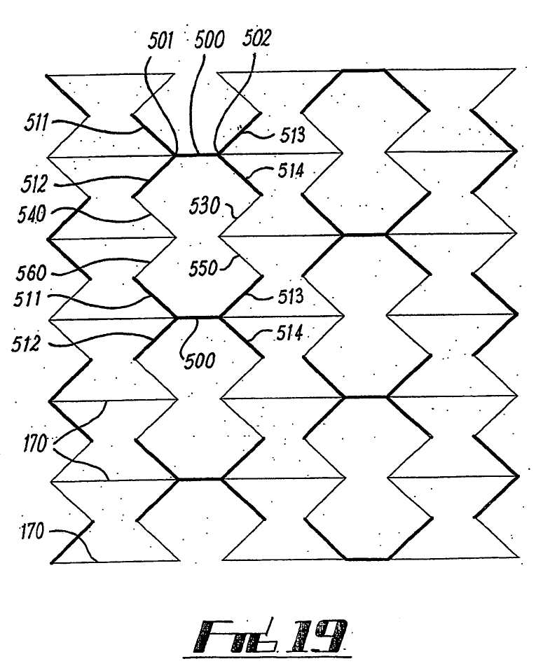

FIG. 17 shows a perspective view of a section of auxetic tubular liner of the present invention having a diameter of about 6 mm and with hexagons having first and second sides (which are parallel with and opposite to one another) oriented in the longitudinal axis of the liner. Hexagons are approximately 613 μm in width and 471 μm in height. Wall thickness of the tubular liner is about 150 μm, and total length is about 2 cm;

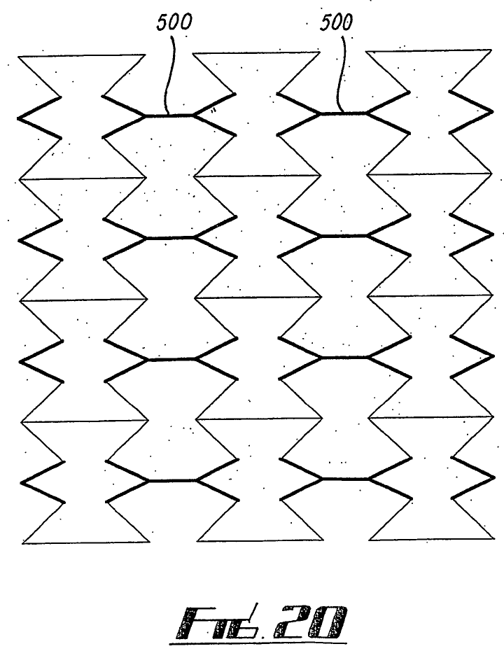

FIG. 18 shows a magnified view of the auxetic tubular liner of FIG. 17; and

FIGS. 19-22 show alternative auxetic structures useful in the auxetic tubular liners of the present invention.

Referring to FIG. 1, this illustrates a typical geometry (inverted hexagons 12 or bow tie honeycomb) which may be micromachined by for example excimer laser technology so as to impart auxetic properties to a substrate material. It will be seen that the application of a tensile load in direction A will result in expansion of the structure in direction B in contrast with conventional materials having a positive Poisson ratio. However, the present invention is not limited to securing auxetic properties by micromachining of geometrical features; such properties may be derived by other methods known in the art, e.g. compression and deformation of polymeric powder particles into a tubular structure under controlled temperature and pressure conditions to produce a material which is, in effect, intrinsically auxetic.

Consideration of the synclastic property of auxetic materials has led the present applicant to the recognition that a tubular liner, e.g. a stent for implantation in an in vivo duct, may be readily inverted or turned inside out Expansion and inversion of a compressed stent initially retained between a mandrel and sleeve results in the release of energy into the plaque (or other blockage) when it is contacted by the inverted stent, thus effecting e.g. dilation of the plaque. This effect is illustrated in FIGS. 2A to 2D. Starting with a relatively short section of a tubular structure 10 having upper and lower ends 14, 16 (FIG. 2A), the structure is compressed laterally, which for the purposes of illustration is supported by a surface underneath its lower surface 16. The structure may be manipulated by releasing the lower end 16, whose diameter as a result increases, while at the same time pressing the upper end 14 towards the support structure (FIG. 2B). For example, this effect is possible if the structure 10 is based on the inverted microhexagon geometry of FIG. 1 so arranged that the sides 11 of the hexagons (i.e. the first and second sides of a hexagon which are parallel with and opposite to one another) are oriented in the circumferential direction with respect to the structure 10, i.e. are oriented perpendicular to the longitudinal axis of the tubular liner. A similar effect can be achieved with hexagons whose first and second sides (which are parallel with and opposite to one another) are oriented in the longitudinal axis of the tubular liner.

Assuming that the material forming the structure 10 is sufficiently flexible, such compression may be continued until the upper end 14 is drawn towards the plane containing the lower end 16 (see FIG. 2C) thus allowing it to be passed through that plane so that, as shown in FIG. 2D, the tubular structure is inverted or turned inside out and the upper end 14 becomes the lower end 16 and vice versa.

The above inversion effect is exploited in the present invention for the purpose of lining a duct, e.g. inserting a stent into an obstructed or narrowed duct, in that the liner or stent employed is of an auxetic material and is sufficiently flexible that it may be inverted within the confines of the duct. For ease of reference, the invention will be described below in terms of a stent for implantation in a blood vessel but it is to be understood that the invention is not limited to this particular application.

Referring now to FIGS. 3 to 7, stent 20 comprises a tubular structure of material which may be intrinsically auxetic or may have been rendered auxetic by suitable techniques such as micromachining of appropriate geometrical features. The stent 20 is located on a reduced diameter leading portion 22 of a mandrel 24 and is in a compressed state between the portion 22 and an outer sleeve 26. The mandrel 24 and the sleeve 26 are arranged so as to be movable relative to one another and are typically made of a low friction/non-stick material such as polytetrafluoroethylene.

The tip 28 of the mandrel portion 22 is of tapering configuration and initially projects to some extent beyond the leading end of the sleeve 26. The assembly comprising the mandrel, stent and sleeve is, in use, coupled to a catheter device so that it can be introduced in the usual manner and positioned in the vicinity of an obstruction or narrowing of the blood vessel. The arrangement is such that the user may operate the assembly through the catheter device to effect movement of the mandrel 24 relative to the sleeve 26 as desired.

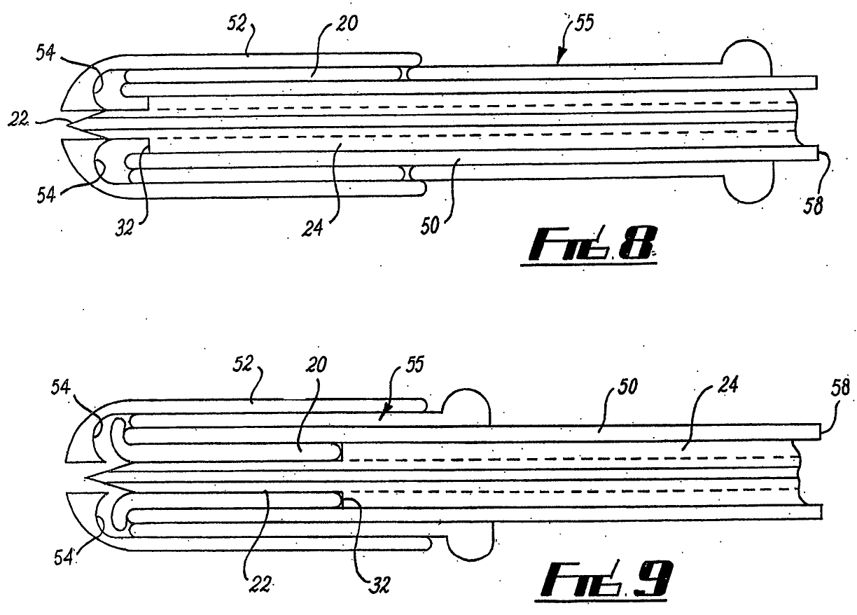

Initially or at some point during the procedure, the leading end of the stent 20 projects beyond the leading end of the sleeve 26 and by virtue of its auxetic properties tends to curl around that end in the manner illustrated in FIG. 6. To facilitate this, the end face 29 of the sleeve 26 is convexly curved.

Once the assembly has been positioned close to the site of obstruction or narrowing of the duct 31 (see plaque deposits 30 in FIGS. 5 to 7) with the aid of a catheter, the mandrel 24 can be manipulated to move forwardly relative to the sleeve 26 so that the stent 20 is advanced forwardly also through its contact with shoulder 32 at the junction between mandrel portion 22 and the remainder of the mandrel. By progressive manipulative operations of the mandrel and sleeve, the stent 20 can be caused to begin inverting so that it folds back over the exterior of the sleeve 26. At the same time, as the stent passes out of the gap between the mandrel portion 22 and the sleeve 26, it is no longer subjected to compression and because of its auxetic properties, it can expand and exert lateral pressure so as to dilate the vessel. In this manner, the stent can be transferred from the assembly into the blood vessel and expand and exert pressure on the plaque or deposit to reduce the obstruction or narrowing (see FIG. 7). Eventually after the stent 20 has been fully deployed within the blood vessel, the mandrel 24 and sleeve 26 may be withdrawn with the aid of the catheter leaving the stent in situ.

Upon self-expansion, the stent forms a region of relatively high curvature during the time that it is undergoing inversion. The resulting “travelling” curved front affords the potential for exerting a sufficiently high pressure to flatten any lesion or further flatten it after pre-dilation.

To facilitate pre-dilation of the duct and thereby assist lining up of the stent during deployment, the mandrel 24 may be designed so that, in the region of its leading end, it may be radially expanded. This can be implemented by providing the mandrel with a central rod 34 which extends through a longitudinal passageway in the mandrel and which has its leading end captive with the leading end of the mandrel portion 22. A section 38 of the portion 22 is formed with a cavity 36 (see FIG. 4) and the walls of the portion 22 is provided with a number of longitudinal slits or apertures (not illustrated) so that this section 38 of the portion 22 can be caused to expand radially by pulling the rod 34 backwards in direction C relative to mandrel 24. When the mandrel is displaced forwardly of the sleeve 26 so as to expose the slitted or apertured section 38, expansion of the section 38 can be effected by manipulation of the rod 34 and mandrel 24 and this can be used to pre-dilate the deposit or plaque 30 to some extent in the artery or duct. One form of rod 34 is a quartz fibre optic catheter through which radiation, e.g. near-ultraviolet radiation from an excimer laser, may be transmitted to the leading end of the mandrel to treat the deposit or plaque material obstructing the artery or the like.

Another feature that may be employed is to provide the mandrel with a longitudinal passageway through which fluidised material (e.g. created by heating or laser treatment of the deposit) can be withdrawn or through which blood flow can be facilitated during stent deployment. In the embodiment illustrated in FIG. 4, this is implemented by using a hollow rod 34 having holes 40 at its distal end to allow fluid entry into the passageway within the rod. Some of the holes may be provided in registry with the cavity 36 so that fluidised material entering via the longitudinal slits or apertures of section 38 can be drawn into the interior of the hollow rod 34.

In a modification as illustrated in FIG. 3 by phantom lines, the mandrel 24 may be telescopic with the portion 22 forming an inner section 22A telescopically received within an outer section 24A of the mandrel. so that the inner and outer mandrel sections can be displaced relative to one another when it is convenient to do so, e.g. during stent deployment or during fabrication of the assembly comprising the stent, mandrel and sleeve (as described below with reference to FIGS. 8 and 9). This arrangement may for instance be employed, in conjunction with the expansion feature described with reference to FIG. 4, to facilitate back-folding of the initial part of the stent around the leading end of the sleeve 26.

In another modification, as discussed hereinbefore, a pathway or pathways may be provided for fluid flow from one end of the assembly to the other so that, for example, blood may flow through the assembly from a location upstream of the narrowing or obstruction in an artery to a location downstream thereof. The fluid flow pathway(s) may for instance be provided by the provision of strategically located apertures or slits in the sleeve 26 and the mandrel 22, 24.

Referring now to FIGS. 8 and 9, the production of the assembly comprising the compressed stent 20, the mandrel 24 and the sleeve 26 is illustrated. Initially the tube 20 of auxetic material is manufactured around a tubular former 50 which is assembled with the mandrel 24 and a housing 52. The housing 52 functions in extruder-like fashion and has an internal curved end face 54 acting as a guide for transfer of the auxetic tube from the former 50 onto the mandrel portion 22. A plunger 55 is assembled to the former 50 (see FIG. 8) and is advanced forwardly to displace the auxetic tube 20 and “extrude” it out of the gap between the former 50 and the housing 52 and onto the mandrel portion 22 (see FIG. 9). At the same time, the mandrel 22 is displaced so that the tube 20 locates on to the mandrel section 22 with one end of the tube 20 immediately adjacent the shoulder 32. Once the tube 20 has been transferred to the mandrel, the housing 52 may be removed and the sleeve 26 is used to displace the former 50 by abutting the leading end of the sleeve 26 against the trailing end 58 of the former and moving the sleeve 26 forwardly to slide the former 50 over the auxetic tube 20 until the sleeve 26 is substituted for the former 50. In this way, the auxetic tube forming the stent 20 is located, in a compressed state, between the mandrel portion 22 and the sleeve 26.

It is envisaged that the double curvature property of auxetic materials will confer advantages relative to conventional metal or metal-based stents in that stent removal by mechanical manipulation may be facilitated without damaging the surrounding artery.

The auxetic nature of the tubular liners of the present invention is shown in FIG. 10, which shows sections of an auxetic tubular liner of the present invention. The sides of the hexagons at (A), (B) and (C) remain the same length. Vertical (radial) compression effects an approximately 13% longitudinal compression and an approximately 40% circumferential compression comparing (A) to (C), equating to an approximate 64% radial compression. The general nature of auxetic structures (as used in the present invention) means that compressing the tubular liner radially will cause a longitudinal compression (shortening). Similarly, a longitudinal expansion (lengthening) will cause a radial expansion. This ability to compress and expand means that the auxetic tubular liners of the present invention are also highly flexible, and expansion of a tubular liner which also causes longitudinal expansion can aid in effecting an inversion of the tubular liner.

FIG. 11 shows a section of an auxetic tubular liner having first and second ends (not shown) defining a longitudinal axis between them, and having a first inverted hexagon structure comprising a plurality on inverted hexagons 100. Each hexagon 100 has: first and second sides 101,102 parallel with and opposite one another; third and fourth sides 103,104 depending from first side 101; fifth and sixth sides 105,106 depending from second side 102. Fourth side 104 is connected to sixth side 106 at second vertex 110, and third side 13 is connected to fifth side 105 at first vertex 120. First side 101 of each hexagon 100 makes an internal angle alpha of less than 90 degrees with each of sides 103,104 and, and second side 102 of each hexagon 100 makes an internal angle alpha of less than 90 degrees with each of sides 105,106.

Sides 101,102 are oriented in the longitudinal axis of the tubular liner.

Each hexagon 100 is connected to first and second adjacent hexagons. Thus for example first side 101 of hexagon 100 comprises a second side of first adjacent hexagon 130, and second side 102 comprises a first side of second adjacent hexagon 140.

The connected hexagons define radial loops 150,160 of interconnected hexagons, the adjacent radial loops being connected by a plurality of connecting members 170.

The exact orientation and arrangement (i.e. positioning) of the connecting members 170 varies between different embodiments of the invention. In this one, a connecting member 170 connects hexagon 100 with hexagon 200 having first and second sides 201,202 parallel with and opposite to one another, third and fourth sides 203,204 depending from first side 201, and fifth and sixth sides 205,206 depending from second side 202. Fourth side 204 is connected to sixth side 206 at second vertex 210.

Connecting member 170 connects hexagons 100,200 between first vertex 120 and second vertex 210.

Each of sides 101,102 is approximately 41 μm wide. The distance between sides 101,102 is approximately 430 μm. Sides 101,102 are approximately 613 μm in length. Sides 103-106 are approximately 30 μm wide, hence their flexibility relative to sides 101,102. The distance between vertices 110,120 is approximately 118 μm. Angle alpha is approximately 46.85 degrees. There are a total of 40 hexagons 100 per circumference of the tubular liner.

Variation in thickness of the tubular liner can be used to e.g. modify its flexibility.

Such inverted hexagon structures provide additional advantages over prior art vascular stents. In particular, the tubular liner of the present invention may act as an embolic containment device, helping to prevent the release of embolic particles into the bloodstream which is a high risk with balloon angioplasty.

In FIG. 12, the same general structure as shown in FIG. 11 is used, albeit oriented perpendicularly to the longitudinal axis of the tubular liner.

Thus, FIG. 12 shows a section of an auxetic tubular liner having first and second ends (not shown) defining a longitudinal axis between them, and having a first inverted hexagon structure comprising a plurality on inverted hexagons 100. Each hexagon 100 bas: first and second sides 101,102 parallel with and opposite one another; third and fourth sides 103,104 depending from first side 101; fifth and sixth sides 105,106 depending from second side 102. Fourth side 104 is connected to sixth side 106 at second vertex 110, and third side 13 is connected to fifth side 105 at first vertex 120. First side 101 of each hexagon 100 makes an internal angle alpha of less than 90 degrees with each of sides 103,104 and, and second side 102 of each hexagon 100 makes an internal angle alpha of less than 90 degrees with each of sides 105,106.

Sides 101,102 are oriented perpendicular to the longitudinal axis of the tubular liner.

Each hexagon is connected to at least a first adjacent hexagon. Thus first side of hexagon 130 is connected to second side 102 of hexagon 100.

Hexagons 100 define longitudinally elongate strips 400,410,420. Thus longitudinally elongate strip 410 is connected to first and second radially adjacent longitudinally elongate strips 400,420 by a plurality of connecting members 170. As mentioned above, the orientation and positioning of connecting members varies between different embodiments of the invention.

In the case of the auxetic tubular liners of FIG. 12, hexagon 250 of radially adjacent strip 400 comprises fourth and sixth sides 254,256 joined at vertex 260. Hexagon 300 of radially adjacent strip 420 comprises fourth and sixth sides 303,305 joined at vertex 320. Connecting member 170 joins vertex 120 to vertex 160, and another connecting member 170 joins vertex 110 to vertex 320.

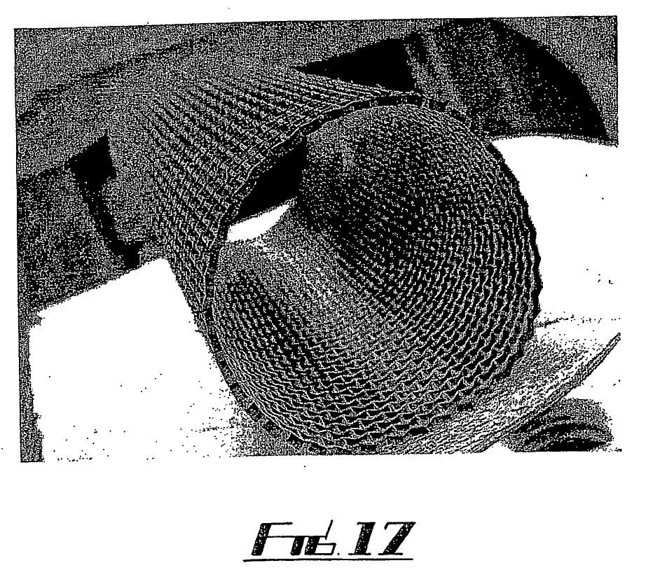

In both of the above cases, connecting members 170 are parallel to the first and second sides. In other embodiments shown in FIGS. 13 and 14, different arrangements of straight connecting members 170 are shown. In FIGS. 15 and 16, non-straight connecting members are used. Specifically, connecting members 171 are angled, and connecting members 172 are curved.

In the case of non-straight connecting members which are capable of flexing in response to force exerted upon them, in order for the structure of the tubular liner to be auxetic then the flexing of the connecting members must not be such that it results in non-auxetic properties. For example, an angled connecting member with a large total length (for example having a single vertex with a small angle) and which is highly flexible could deform upon the exertion of pressure such that the structure was not auxetic. Conversely, an angled connecting member with a shorter total length, and which is much less flexible (possibly having a single vertex making a larger angle) will be less flexible and therefore the structure may remain auxetic. The same basic principle also applies to other non-straight connecting member shapes (e.g. curves).

In certain embodiments of the present invention, the adjacent loops of hexagons are arranged such that they are offset relative to one another, e.g. with a first loop arranged so that the vertices of its first and second sides with its third and fifth sides are proximal to the vertices made between the fourth and sixth sides of hexagons of a second loop (or adjacent strip) of hexagons.

FIGS. 17 and 18 show auxetic tubular liners made according to the present invention, and which are capable of being inverted e.g. using a mandrel as described above. The tubular liners are fabricated from nylon tubing (although other materials such as e.g. polyurethanes and others as discussed above can be used) made by taking a tube and placing a mask over a section of the tube, the mask having a structure cut into it which is a negative of the desired structure of the tubular liner. An excimer laser is then used to etch (ablate) the pattern defined by the mask from the tube, thus leaving a section of the desired auxetic structure. The mask is then moved and the process repeated to extend the pattern etched from the tube and produce an auxetic tubular liner having a desired structure. A wide range of parameters for the excimer laser are available, for example energy density and frequency, and focal length. Other parameters such as mask size, ablation ratio, and material of the tube can also be altered in order to achieve optimum results. In some cases the generation of plasma by a laser beam impacting a surface being etched results in small “rests” being left on the resulting structure. An ultrasonic bath can aid in the removal of any “rests”, should that be necessary or desired.

Generally, since the excimer laser is used to cut the auxetic structure into the e.g. polyurethane materials, the auxetic structure has a predefined or natural set of dimensions to which it will tend. It can, of course, be expanded or compressed, and also inverted. However, it will always tend back to the original dimensions of the tubular liner material.

In addition, certain embodiments of the present invention have first and second sides parallel with and opposite to one another replaced with thick sides having relatively inflexible thick branched sections extending from them. In such cases, the resulting polygons can still be considered to be the above “hexagons”, albeit with their first and second sides replaced with structures which although not straight do not detract from the auxetic nature of the structure. Importantly, the third, fourth, fifth and sixth sides remain flexible such that they can modify their conformation/shape and effect auxetic properties for the tubular liner.

As is shown in FIG. 19, first and second sides are replaced with a first side 500 having first and second vertices 501,502, and with first and second arms 511,512 extending from vertex 501 and arms 513,514 extending from second vertex 502, each of first and second arms 501-504 making an internal angle with first side 500 of between 90 and 180 degrees (in the case shown, approximately 135 degrees). Third, fourth, fifth and sixth sides 530,540,550,560 depend from the first and second arms of the first and second sides, thus completing the polygons. Sides 530-560 are relatively flexible compared to the first and second sides 500 and arms 511-514, ensuring the auxetic properties of the structures and tubular liners.

FIG. 19 also shows that it is possible for connecting members 170 to connect vertices of the first and second sides 500 with e.g. vertices made between third and fifth sides, or fourth and sixth sides, and for the resulting structure to be auxetic. Notably, there is no connection of a first or second side with an adjacent first or second side of an adjacent hexagon/polygon.

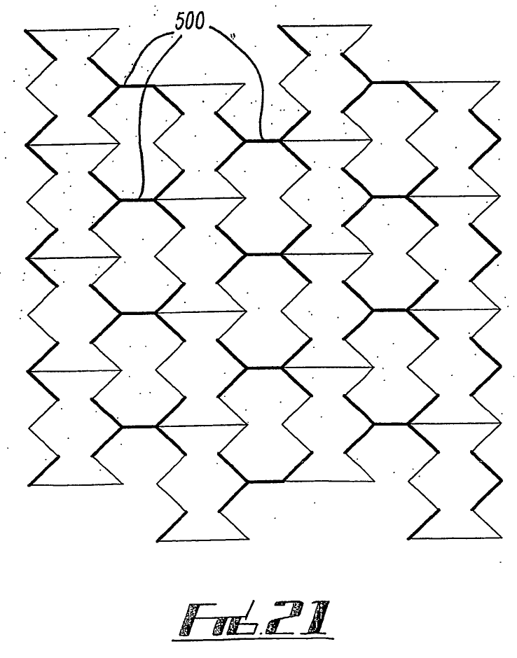

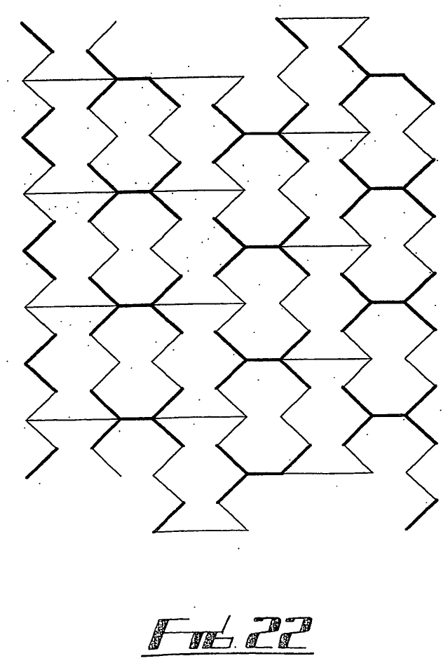

The structures shown in FIGS. 20-22 are also auxetic, and can also be used in the present invention. As is shown in FIGS. 21 and 22, structures which have connecting members joined to the vertices of the first and second sides can be auxetic, in this case joining the vertices of the first and second sides to the vertices between the third and fifth, and fourth and sixth sides. The structure shown in FIG. 21 is more auxetic than that shown in FIG. 22 since a greater proportion of the connecting members 170 are able to move relative to the first and second sides of adjacent hexagons.

Whilst endeavouring in the foregoing specification to draw attention to those features of the invention believed to be of particular importance, it should be understood that the Applicant claims protection in respect of any patentable feature or combination of features disclosed herein and/or shown in the drawings whether or not particular emphasis has been placed on such feature or features.

1. A tubular liner for insertion into a duct, said tubular liner defining first and second ends and a lumen, both of said first and second ends being open, such that fluid flow can occur through said tubular liner from said first end to said second end, characterised in that said liner comprises an auxetic material.

2. A tubular liner according to claim 1, defining a longitudinal axis between said first and second ends, having a structure comprising a plurality of adjacent radial loops arranged about said tubular liner, each radial loop comprising a plurality fo interconnected hexagons having:
(i) first and second sides parallel with and opposite to one another;
(ii) third and fourth sides dependent from said first side; and
(iii) fifth and sixth sides dependent from said second side;
said third side being connected to said fifth side at a first vertex, and said fourth side being connected to said sixth side at a second vertex;
said first side of each hexagon having an internal angle of less than 90 degrees with each of said third and fourth sides, and said second side making an internal angle of less than 90 degrees with each of said fifth and sixth sides;
said first and second sides of said hexagons being oriented in said longitudinal axis;
each hexagon being connected to first and second adjacent hexagons, said first side of each hexagon comprising a second side of said first adjacent hexagon, and said second side comprising a first side of said second adjacent hexagon;
each radial loop being connected to at least a first adjacent radial loop, each pair of first and second adjacent radial loops being connected by a plurality of connecting members.

3. A tubular liner according to claim 2, said plurality of connecting members being between third and fifth sides of said plurality of hexagons of said first loop and said fourth and sixth sides of said plurality of hexagons of said second loop.

4. A tubular liner according to claim 3, said connecting members being between said first vertex of said hexagons of said first loop and said second vertex of said hexagons of said second loop.

5. A tubular liner according to claim 1, defining a longitudinal axis between said first and second ends, having a structure comprising a plurality of longitudinally elongate strips fo interconnected hexagons oriented along said longitudinal axis of said tubular liner, each longitudinally elongate strip comprising a plurality of interconnected hexagons having:
(i) first and second sides parallel with and opposite to one another;
(ii) third and fourth sides dependent from said first side; and
(iii) fifth and sixth sides dependent from said second side;
said third side being connected to said fifth side at a first vertex, and said fourth side being connected to said sixth side at a second vertex;
said first side of each hexagon making an internal angle of less than 90 degree with each of said third and fourth sides, and said second side making an internal angle of less than 90 degrees with each of said fifth and sixth sides;
said first and second sides of said hexagons being oriented perpendicular to said longitudinal axis;
each hexagon being connected to at least a first adjacent hexagon, said first side of each hexagon comprising a second side of said first adjacent hexagon, and said second side comprising a first side of any second adjacent hexagon;
each longitudinally elongate strip being connected to first and second radially adjacent longitudinally elongate strips by a plurality of connecting members.

6. A tubular liner according to claim 5, said plurality of connecting members being between:
(a) said third and fifth sides of said plurality of hexagons of said longitudinally elongate strip and said fourth and sixth sides of said plurality of hexagons of said first radially adjacent longitudinally elongate strip; and
(b) said fourth and sixth sides of said plurality of hexagons of said longitudinally elongate strip and said third and fifth sides of said plurality of hexagons of said second radially adjacent longitudinally elongate strip.

7. A tubular liner according to claim 6, said connecting members being between:
(a) said vertex of said hexagons of a given longitudinally elongate strip and said second vertex of said hexagons of a first radially adjacent longitudinally elongate strip of hexagons; and
(b) said second vertex of said hexagons of said given longitudinally elongate strip and said first vertex of said hexagons of a second radially adjacent longitudinally elongate strip of hexagons.

8. A tubular liner according to any of claim 2, said connecting member having a shape selected from the group consisting of: straight, curved and angled.

9. An assembly for use in lining a section of duct, said assembly comprising:
(i) a tubular liner according to claim 1;
(ii) a mandrel upon which said auxetic tubular liner is located; and
(iii) a sleeve surrounding said mandrel and auxetic tubular liner, said sleeve having and open end;
said mandrel being movable relative to said sleeve.

10. The use of a tubular liner according to claim 1 in the manufacture of an assembly according to claim 9 for use in lining a section of duct.

11. A method of manufacture of a tubular liner according to claim 2 from a tube defining first and second open ends and a lumen, said tubular liner being located on a mandrel, said method comprising the steps of:
(i) placing over a region of said tube an etching mask defining at least a part of said structure of said tubular liner; and
(ii) etching said tube through said mask to define said mask structure on said tube;
and optionally performing at least once the step of:
(iii) moving said mask relative to said tube and repeating steps (i) and (ii) to define an additional region of said mask structure on said tube.

12. A method of inserting a tubular liner according to claim 1 into a duct, said tubular liner defining first and second faces, said first face facing said lumen, said second face facing away from said lumen, said method comprising the steps of:
(i) locating said tubular liner on a mandrel surrounded by a sleeve to define an assembly, said sleeve having an open end;
(ii) passing said assembly into said duct;
(iii) moving said mandrel relative to said sleeve so as to cause said tubular liner to be displaced through said sleeve open end such that said tubular liner folds back over said sleeve and inverts within the confines of said duct such that said second face faces said lumen of said inverted tubular liner and said first face faces away from said lumen of said inverted tubular liner;
(iv) withdrawing said sleeve and said mandrel from said duct, leaving said inverted tubular liner in situ.
